(19) United States
(12) Patent Application Publication
(10) Pub. No.: US 2013/0315830 A1
(43) Pub. Date: Nov. 28, 2013

(54) PSMA as a BioMarker for Androgen Activity in Prostate Cancer

(71) Applicant: Cornell University, (US)

(72) Inventor: Neil H. BANDER, Chappaqua, NY (US)

(73) Assignee: Cornell University, Ithaca, NY (US)

(21) Appl. No.: 13/778,306

(22) Filed: Feb. 27, 2013

Related U.S. Application Data
(60) Provisional application No. 61/604,039, filed on Feb. 28, 2012.

(51) Int. Cl. G01N 33/574; A61K 49/00

(52) U.S. Cl. CPC G01N 33/57492; A61K 49/00
USPC 424/9.1; 435/7.24; 424/174.1

(57) ABSTRACT

The androgen receptor (AR) is the key driver of prostate differentiation and prostate cancer (PC) progression, and androgen ablation is the cornerstone of advanced PC treatment. Prostate-specific membrane antigen (PSMA) represents another target of interest in PC. Previous publications have reported inconsistent associations between androgen levels and PSMA expression. Using a panel of prototypical human PC cell lines, this relationship is clarified. PSMA is a biomarker that distinguishes AR-positive/PSMA-positive adenocarcinomas from AR-negative variants. PSMA is a cell surface barometer of androgen activity that can be readily identified by immunohistochemistry and/or in vivo imaging. Given that anti-androgen therapy is likely to remain a cornerstone of PC treatment, the associated up-regulation of PSMA, as well as its other characteristics, makes it a compelling target opportunity in PC.

This application claims priority to U.S. Provisional Patent Application No. 61/604,039, filed Feb. 28, 2012, the entirety of which is hereby incorporated by reference.

### BACKGROUND

The androgen receptor (AR) is the key driver of prostate epithelial differentiation and prostate cancer (PC) progression, and androgen ablation is the cornerstone of advanced PC treatment. Recently, more potent anti-androgenic agents capable of virtual complete suppression of endocrine and intracrine androgen synthesis and signaling have demonstrated clinical benefit (Morris et al., JCO 2010; 28(9): 1496-1501; and Scher et al. Lancet 2010; 375:1437-46). But many patients manifest de novo or acquired resistance to these therapies, suggesting the continuing need to develop additional therapeutic targets and agents. And currently, the only biomarker utilized to measure androgen action is serum prostate specific antigen (PSA); there is no way to measure androgen axis activity at the level of the cell or lesion in vivo.

Prostate-specific membrane antigen (PSMA) represents another molecule of interest in PC. PSMA has many features that make it an attractive and valuable target: (1) its expression is highly specific for prostatic epithelium; (2) it is significantly up-regulated in PC (Israeli et al., Can Res 1994; 54:1807-11; Wright et al., Urol Oncol 1995; 1:118-28; Troyer et al., Int J Can 1995; 62: 552-58; and Sokoloff et al., The Prostate 2000; 4: 3150-57); (3) it is expressed by virtually all PCs (Wright et al., Urol Oncol 1995; 1:118-28; Sweat et al., Urol 1998; 52: 637-40; Bostwick et al., Cancer 1998; 82: 2256-61; Mannweiler et al., Pathol Oncol Res 2009; 15: 167-72; Kusumi et al., Pathol Int 2008; 58: 687-94; Ananias et al., Prostate 2009; 69: 1101-8); (4) its expression increases directly with tumor grade and clinical aggressiveness (Wright et al., Urol Oncol 1995; 1:118-28); and (5) it functions as an internalizing cell surface receptor. Previous publications have run the gamut with respect to showing an association between androgen levels and PSMA expression. On the one hand, Israeli (Israeli et al., Can Res 1994; 54:1807-11) noted PSMA down-regulation in the LNCaP cell line in the presence of androgens and Wright (Wright et al., Urology 1996; 48:326-334) found that 55% (11 of 20) of primary PCs expressed higher levels of PSMA after hormonal therapy. On the other hand, Chang reported no increase in PSMA expression when comparing prostatectomy specimens from patients undergoing 3 months of neo-adjuvant androgen ablation versus those who did not (Chang et al., Cancer 2000; 88: 407-415). Kusumi reported that PSMA expression was decreased by hormonal therapy and, noting the conflicting literature, suggested further study was necessary (Kusumi et al., Pathol Int 2008; 58: 687-94).

Given that androgen inhibition will almost certainly remain a critical component of any PC therapy plus previous observations of a possible relationship between androgen activity and PSMA expression, clarification of this relationship was needed.

### BRIEF SUMMARY OF THE INVENTION

When a panel of prototypical human PC cell lines was examined, PSMA expression consistently co-typed with those lines that were AR-positive, some of which also expressed PSA. Two prototypic AR-negative lines, PC3 and DU145, were PSMA-negative and PSA-negative. In this panel of cell lines, PSMA was tightly and more faithfully linked to AR expression than PSA. This suggests that classical prostatic adenocarcinoma bears the phenotype of AR⁺, PSMA⁺, PSA^{+/-} whereas the triple-negative (AR⁻, PSMA⁻, PSA⁻) phenotype is characteristic of small cell or other variants (Tai et al., Prostate 2011; 71:1668-1679; and Beltran et al., Cancer Discovery 2011; 1:487-495). This in vitro finding is consistent with multiple publications that report PSMA expression by approximately 95% of PC cases (Wright et al., Urol Oncol 1995; 1:118-28.; Sweat et al., Urol 1998; 52: 637-40; Bostwick et al., Cancer 1998; 82: 2256-61; Mannweiler et al., Pathol Oncol Res 2009; 15: 167-72; Kusumi et al., Pathol Int 2008; 58: 687-94; and Ananias et al., Prostate 2009; 69: 1101-8), reflecting the known preponderance of adenocarcinomas relative to small cell variants. PSMA, therefore, is a biomarker that can be easily identified by immunohistochemistry, circulating tumor cell (CTC) analysis, and/or in vivo imaging to identify and distinguish AR-positive/PSMA-positive adenocarcinomas from AR-negative variants.

It was discovered that androgen withdrawal from AR-positive lines, in all cases, led to as much as a 10-fold increase in PSMA expression relative to its level in physiological concentrations of DHT. Similarly, silencing the AR gene, in all cases, led to increased PSMA expression whereas increasing AR expression via transfection led to decreased PSMA expression. These findings suggest that the effect of androgen withdrawal on PSMA is mediated via AR and that PSMA is an AR-regulated (repressed) gene. It is likely that liganded AR suppresses PSMA expression via its known binding to regulatory sequences of the PSMA gene (Noss et al., Gene 2002; 285(1-2):247-256; Watt et al., Genomics 2001; 73:243-54; and Evans et al., PNAS 2011; 108:9578-9582). If AR activity is decreased due to pathophysiologic or pharmacologic reasons, the PSMA gene is de-repressed. Nevertheless, while the directional changes in PSMA expression associated with changes in androgen level were identical among the different lines, the various cell lines expressed widely different levels of PSMA even under identical concentrations of DHT. This suggests that PSMA expression is not a first order stoichiometric event based purely on androgen level but other factors are involved. PSMA, nevertheless, represents a useful cellular biomarker to aid in interrogating AR gene regulation. A static reading of PSMA level will be less informative than a comparison of readings pre- and post-therapeutic intervention. Importantly, our findings indicate that the time to peak PSMA expression is approximately 2 weeks after complete hormonal withdrawal. Use of shorter intervals for assessment may cause underestimation of actual hormonal effects. The nature of the therapeutic intervention itself may effect the time to peak expression and should be determined for each form of intervention.

PSA and PSMA both represent biomarkers of androgen activity, albeit the former is stimulated while the latter is suppressed by androgens. In addition, while PSA may be sampled in plasma or serum and represents the average output of all lesions, the absolute level as well as changes in PSMA expression can be used as a pharamcodynamic biomarker of androgen activity at the level of the individual cell or lesion. For example, ex vivo analysis of captured CTCs or in vivo patient imaging with PSMA-targeted agents can identify PSMA up-regulation indicating suppression of androgen activity (Evans et al., PNAS 2011; 108:9578-9582) or vice versa. Lack of PSMA would suggest a non-adenocarcinoma variant particularly if found in association with a low/absent PSA and direct therapies away from hormonal manipulation to more appropriate approaches. Clinical trials using ⁸⁹Zirconium-J591, a PSMA-targeted immunoPET agent capable of quantitative reporting of PSMA levels in vivo, would corroborate these findings (Holland et al., J Nucl Med 2010; 51: 1293-1300).

Lastly, the obligate expression of AR in prostate adenocarcinomas, the tight linkage between AR and PSMA expression, the central role of anti-androgen therapy in PC coupled with the effect of anti-androgen therapy to increase PSMA expression make AR and PSMA a compelling coordinate target combination. This paradigm may be incorporated into PSMA-targeted antibody therapy trials.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a western blot result depicting the co-expression of PSMA and AR by eight cell lines. Six of these lines were AR⁺/PSMA⁺; 1 cell line (PC3) was AR⁻/PSMA⁻. One line (PC3-PSMA) represents the PC3 line transfected with PSMA (Stephan, M. T. et al, Nat Med 2007, 13:1440-1449) was AR⁻/PSMA⁻. DU145 (not shown) was also AR⁻/PSMA⁻.

FIG. 2A shows a western blot result depicting the upregulation of PSMA in the LAPC-4 PC cell line (wild-type AR), of 5.7-fold when grown in charcoal-stripped FCS medium relative to medium supplemented with FCS plus physiological levels of DHT. FIGS. 2B and 2C show charts depicting the up-regulation of PSMA of about 7-8-fold that peaked at 2 weeks in LNCaP and MDA-PCa-2b (both with mutated AR) cell lines that are grown in charcoal-stripping the growth media, respectively. According to FIG. 2B, the level of PSMA up-regulation is minimal at 1 week in relation to 2 weeks. FIG. 2D shows a chart that depicts the dose-response of PSMA expression relative to media steroid concentration; a table that lists the percentage of media used in cell culture, the corresponding mean fluorescence intensity (MFI) and the ratio of MFI value in each sample to the MFI in sample; and a chart that depicts the approximately linear PSMA expression increasely relative to decreasing concentration of steroids in the growth medium.

FIG. 3A shows a chart that depicts the down-regulation of PSMA by approximately 80% by transfectinng AR gene into LNCaP to over-express AR (i.e., LNCaP-AR). FIG. 3B shows a western blot depicting a dose-dependent up-regulation of PSMA by silencing AR with siRNA in cell lines LNCaP and CWR22Rv1, respectively. FIG. 3C shows a western blot depicting a dose-dependent up-regulation of PSMA by silencing AR with siRNA in cell lines MDA-Pca-2b and LAPC-4, respectively.

FIGS. 4A-4C show charts depicting the successful down-regulation of AR by AR siRNA treatement and the corresponding up-regulation of PSMA in cell lines LNCaP (FIG. 4A), MDA-PCa-2B (FIG. 4B) and LAPC-4 (FIG. 4C), respectively. Silencing AR led to a significant decrease in PSA secretion as expected (data not shown). FIG. 4D shows a table listing the exaction numbers detected in each cell line LNCaP, MDA-PCa-2B and LAPC-4 under different treatment regime. For example, AR expression was 16.23 in LNCaP cell line when teated with AR siRNA transfection. Control: untreated group. NT-siRNA transfection: treated with non-targeted siRNA. Secondary antibody alone: as a negative control for detection.

FIG. 5 shows photos depicting immunohistochemical assessment of PSMA expression before and after castration. In panel A, low level expression of PSMA was observed in CWR22Rv1 xenografts growing in intact, androgen-replete nu/nu mice, which was consistent with in vitro findings (see FIG. 1, lane 5). In panels B, C and D, the levels of PSMA expression rose progressively at 1, 2, and 4 weeks subsequent to surgical castration, respectively. This gradual increase of PSMA expression was also consistent with in vitro findings. Similarly, immunohistochemistry reveals significantly elevated PSMA expression in benign prostatic resections of patients treated with 5-α reductase therapy relative to untreated patients (data not shown).

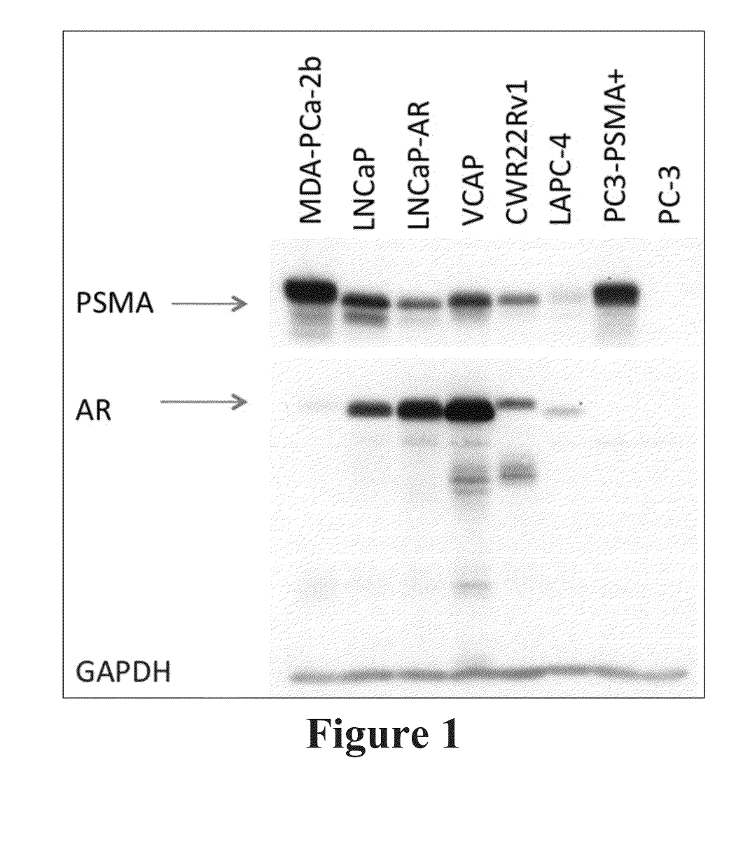

### EXAMPLES

### Expression of PSMA and AR by Eight Cell Lines

For western blots, equal amounts of cell lysates were loaded in each lane. PSMA was detected by monoclonal antibody (mAb) J591; AR was detected by mAb anti-AR (AR441). GAPDH was used as a loading control. Six of the cell lines (MDA-PCa-2b, LNCaP, LNCaP-AR, VCAP, CWR22Rv1 and LAPC-4) expressed both AR and PSMA. MDA-PCa-2b expresses a relatively low level of AR, just barely visible at this exposure. CWR22Rv1 expresses a slightly larger AR protein (114 KD, instead of 110 KD) due to duplication of exon 3). PC3 and another cell line, DU145 (separate gel, not shown), were AR⁻/PSMA⁻. PC3-PSMA is the PC3 line stably transfected with PSMA.

Human prostate cancer cell lines, LNCaP, CWR22Rv1, MDA-PCa-2b and LAPC-4 were purchased from American Type Culture Collection (Manassas, Va.). LNCaP/AR and PC3-PSMA were gifts from Charles Sawyers and Michel Sadelain, respectively (MSKCC, NY). LNCaP, LNCaP/AR, and CWR22Rv1 cells were maintained in RPMI1640 medium supplemented with 2 mM L-glutamine (Invitrogen, Carlsbad, Calif.), 1% penicillin-streptomycin (Invitrogen), and 10% heat-inactivated fetal calf serum (FCS) (Invitrogen). MDA-PCa-2b cells were grown in F12K medium containing 2 mM L-glutamine, 1% penicillin-streptomycin, 20% heat-inactivated FCS, 25 ng/mL cholera toxin (Sigma-Aldrich, St. Louis, Mo.), 10 ng/mL epidermal growth factor (BD Biosciences, San Jose, Calif.), 5 μM phosphoethanolamine (Sigma-Aldrich), 100 pg/mL hydrocortisone (Sigma-Aldrich), 45 nM selenious acid (Sigma-Aldrich), and 5 μg/mL insulin (Sigma-Aldrich). LAPC-4 cells were maintained in IMDM medium supplemented with 2 mM L-glutamine, 1% penicillin-streptomycin and 5% heat-inactivated FCS. All cell lines were kept at 37° C. in a 5% CO2 atmosphere. The 5α-dihydrotestosterone (DHT) was purchased from Wako Chemical USA (Richmond, Va.).

MAb anti-PSMA J591 was generated (Liu et al., Cancer Res 1997; 57: 3629-34). Additional antibody (Ab) reagents included: mAb anti-AR (AR441), Rabbit anti-Human AR and goat polyclonal anti-GAPDH (Santa Cruz Biotechnology, Santa Cruz, Calif.), and mAb anti-PSA (Dako, Glostrup, Denmark). Mouse mAb anti-human beta-Actin was purchased from Thermo Scientific (Rockford, Ill.).

In order to run Western Blots, cells were lysed with Cell Lysis Buffer (Cell Signaling Technology, Danvers, Mass.) containing 1 mM phenylmethylsulphonyl fluoride (EMD Chemicals, Gibbstown, N.J.). Equal amounts of protein were applied in each well on a 10% Tris-HCl gel (Bio-Rad Laboratories, Hercules, Calif.). The proteins were transferred onto Immobilon-P Membranes (Millipore, Billerica, Mass.), after which the filters were probed with the following reagents: murine anti-PSMA mAb J591, murine mAb anti-AR (AR441), rabbit anti-human AR, murine mAb anti-human beta-actin, and/or goat polyclonal anti-GAPDH. For quantitative western blots, the Li-cor Odyssey Infrared Imaging System (Lincoln, Nebr.) was used. With this system, two different proteins of the same molecular weight (e.g., PSMA and AR) can be detected simultaneously and quantified on the same blot using two different antibodies from two different species (mouse and rabbit) followed by detection with two IRDye labeled secondary antibodies. Anti-beta-actin is used as a loading reference. Millipore Immobilon-FL PVDF membranes were used following Licor's recommendations. MuJ591 anti-PSMA 1 ug/ml, rabbit anti-human AR 1:500 and mouse anti-human beta-actin 1:10,000 in dry milk/PBST were combined and incubated simultaneously with the membranes for 1 hour. After washing, IRDye 800CW-goat anti-mouse secondary antibody (1:10,000) and IRDye 680LT-goat anti-rabbit secondary antibody (1:20,000) in 5% dry milk/PBST were combined and incubated simultaneously with the membranes. After washing, the membranes were scanned and the bands were quantified with the Odyssey Infrared Imaging System.

### Androgen Withdrawal Up-Regulates PSMA Expression

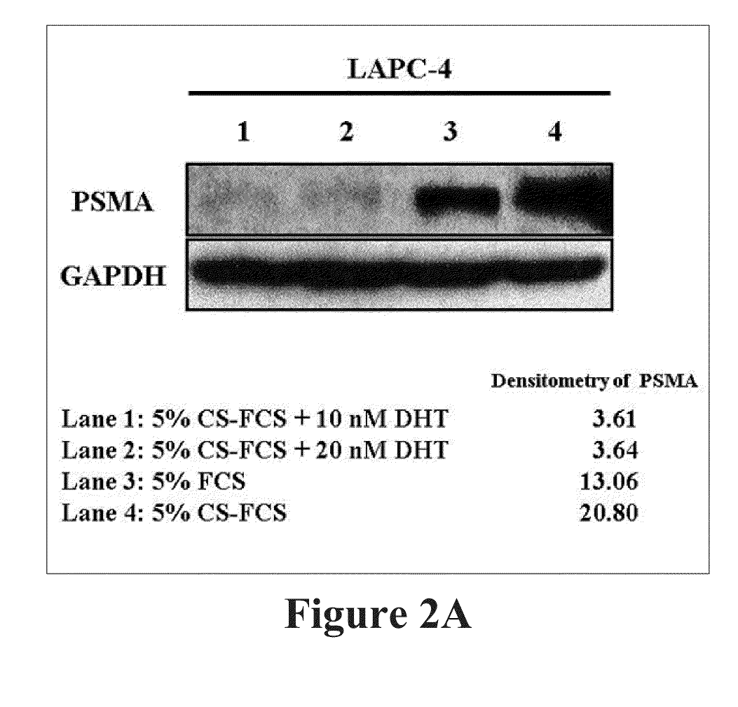

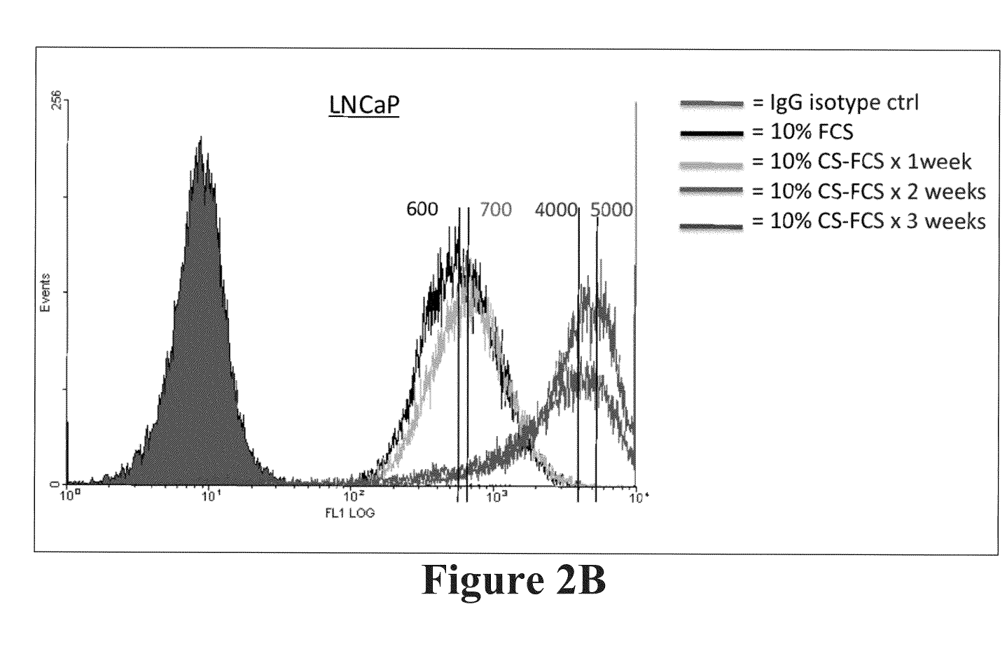

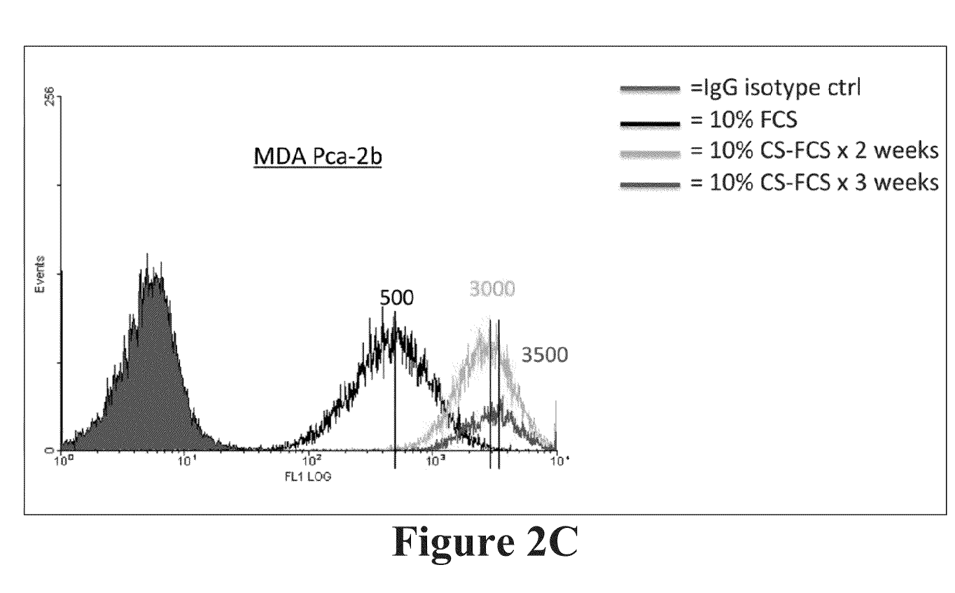

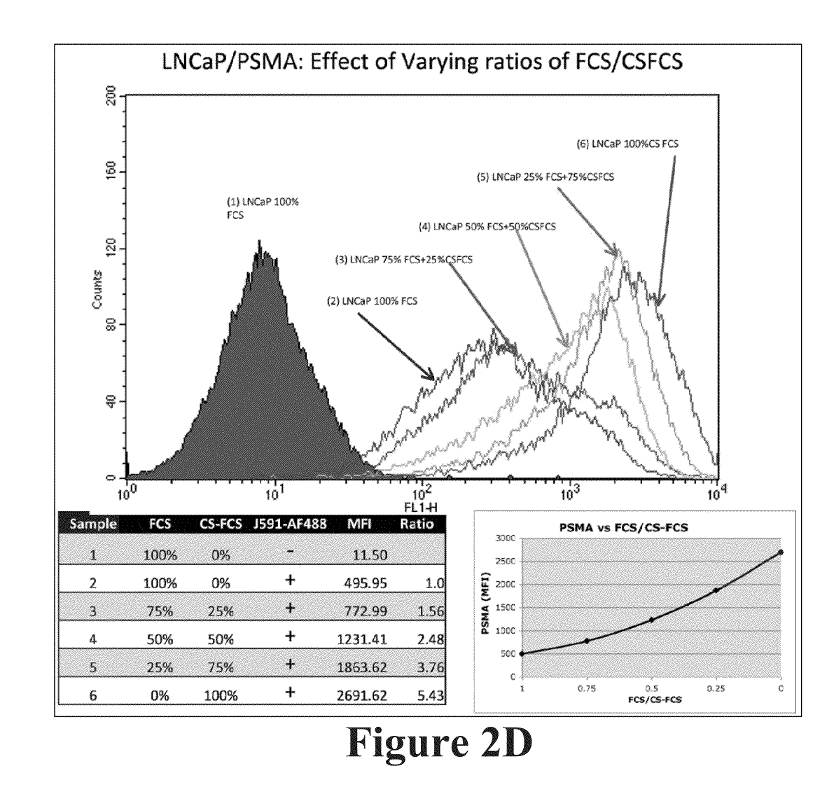

LAPC-4, expressing wild-type AR, grown in physiological levels of DHT (10-20 nM) expresses a low level of PSMA (Lanes 1 and 2) (FIG. 2A). When grown in standard 5% FCS which contains very low levels of androgens, PSMA expression increases 3.6-fold (Lane 3). When the FCS is charcoal-stripped of all steroids, PSMA level rises further, by 5.7-fold (Lane 4) that seen with physiological levels of DHT. FACS analysis demonstrates that LNCaP and MDA-PCa-2b, both with mutated AR, have elevated PSMA levels at baseline in standard FCS-supplemented medium (FIGS. 2B and 2C). Use of charcoal-stripped FCS further up-regulates PSMA 7-9-fold, peaking at 2 weeks. The lower cell number at 3 weeks reflects cell loss from steroid starvation. Dose response of PSMA expression by LNCaP cells grown for 2 weeks with varying levels of androgens (FIG. 2D). Progressive steroid deprivation progressively leads to an increase in PSMA of 5.4-fold.

### PSMA Expression Level is Inversely Related to AR Level

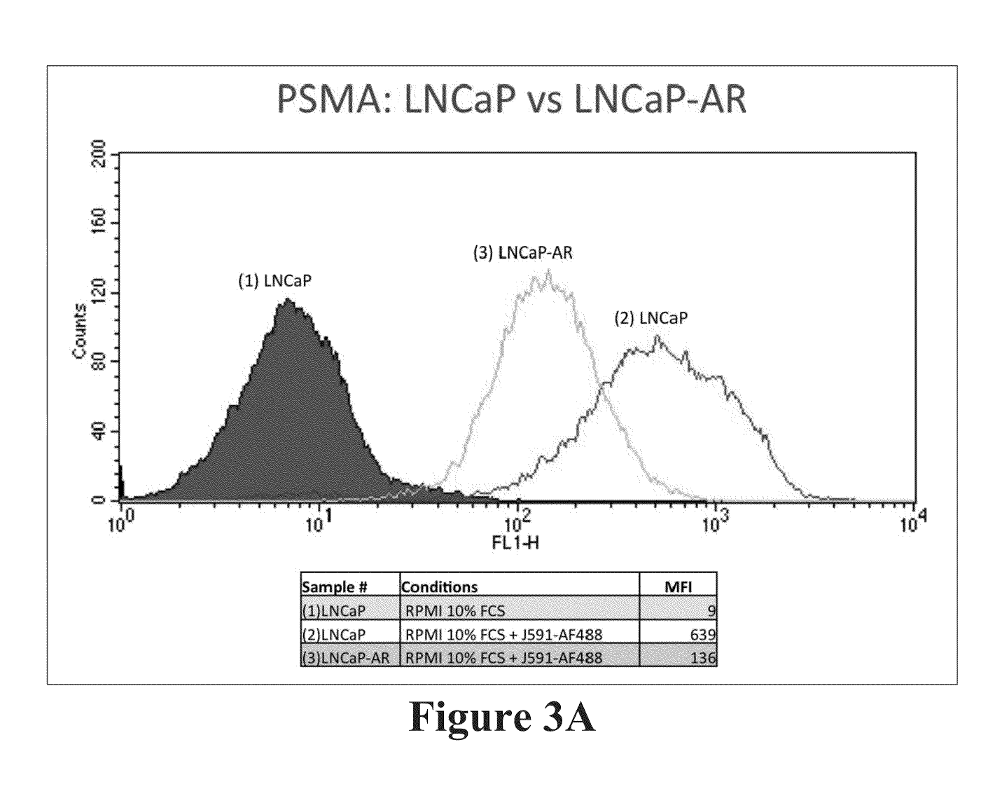

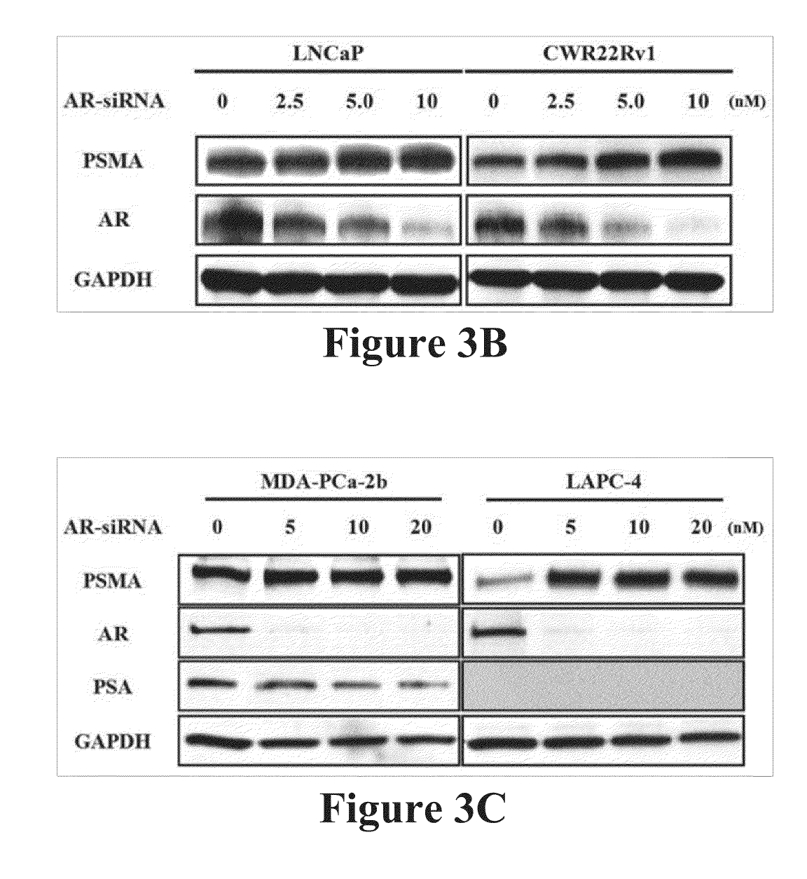

Transfection of AR into LNCaP (LNCaP-AR) results in down-regulation of PSMA expression by approximately 80% as measured by FACS (FIG. 3A). Conversely, AR-siRNA treatment silences AR and up-regulates PSMA expression in LNCaP and CWR22Rv1 at 48 hours (FIG. 3B) and in MDA-PCa-2b and LAPC-4 cells at 4 days (FIG. 3C).

### Silencing AR Up-Regulates PSMA

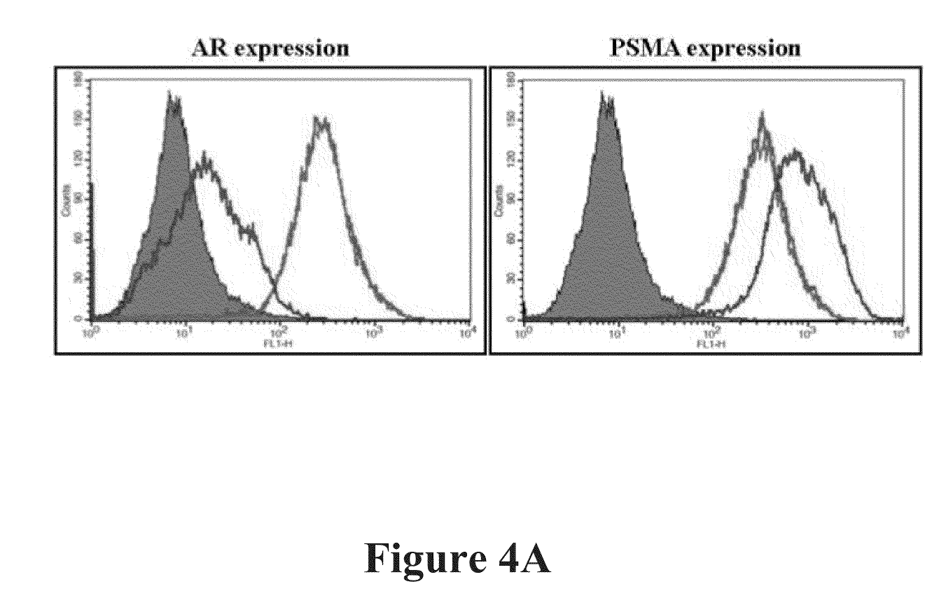

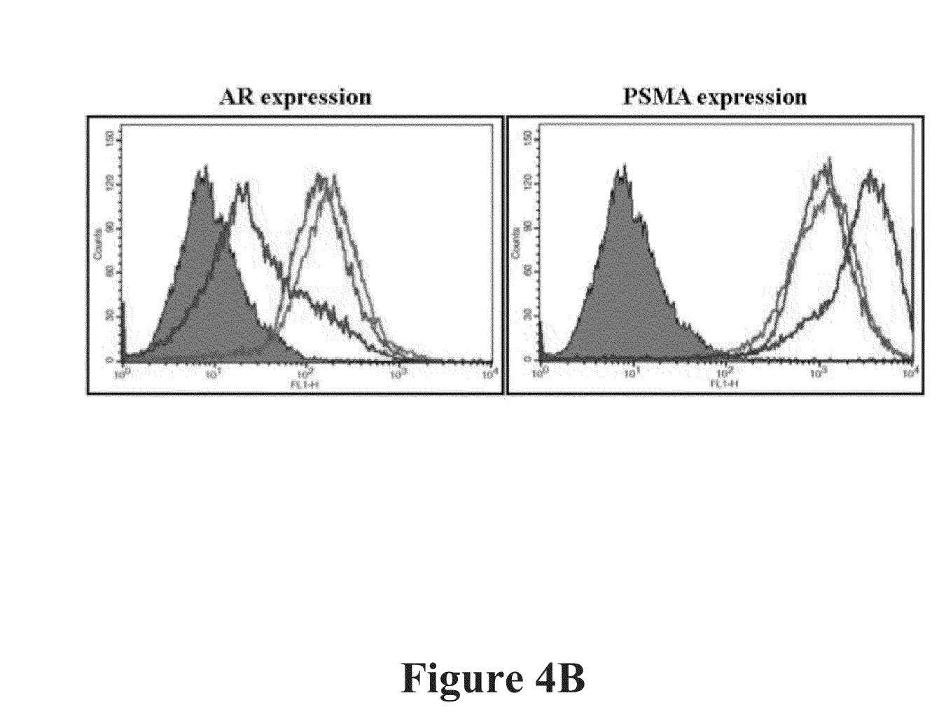

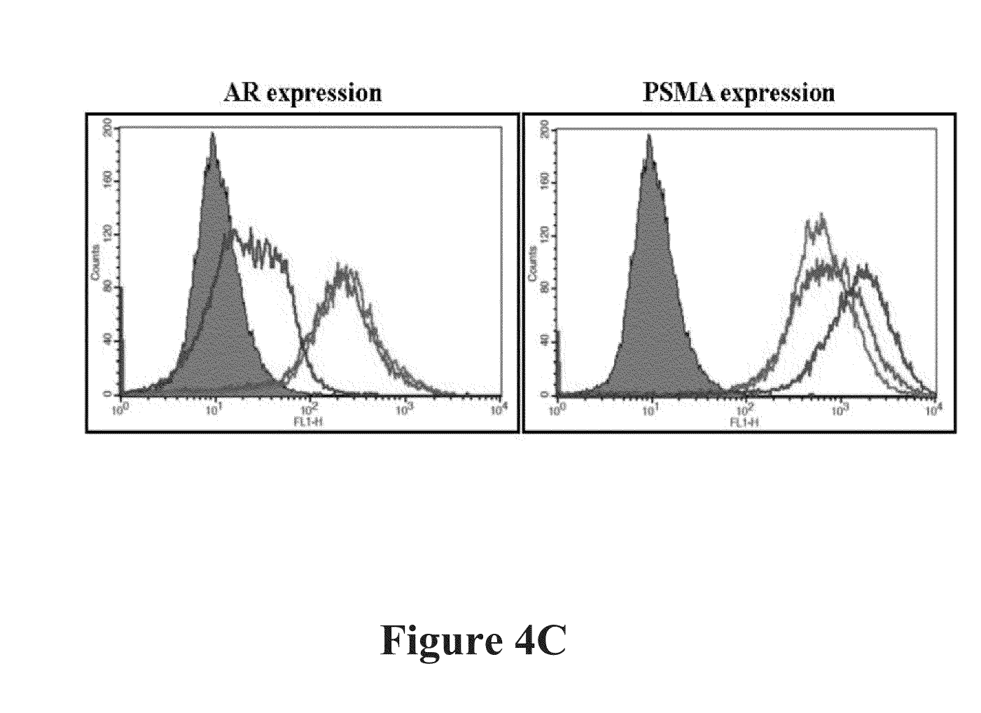

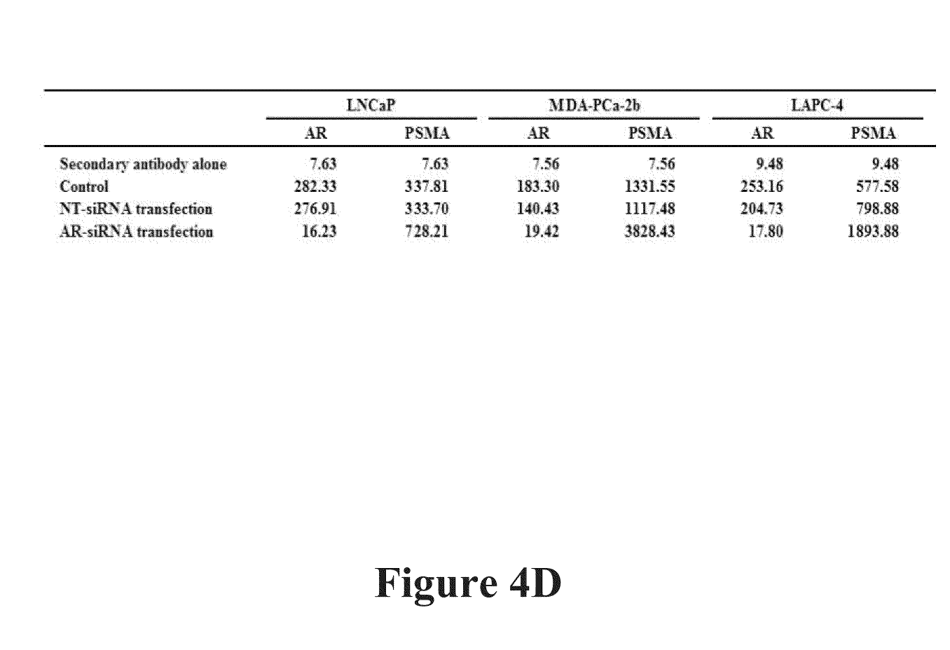

FACS analysis of LNCaP (FIG. 4A), MDA-PCa-2b, (FIG. 4B) and LAPC-4 cells (FIG. 4C) treated with AR-siRNA (blue line), non-targeted-siRNA (red line) and untreated control (green line) was conducted. The gray histogram is secondary antibody-only negative control. In all cases, AR-siRNA silenced AR and up-regulated PSMA; the non-targeted-siRNA control did not affect expression of either AR or PSMA.

Short interfering RNA (siRNA) duplexes specific to AR as well as non-targeting siRNA (NT-siRNA) were purchased from Dharmacon (Lafayette, Colo.). The AR-specific siRNA (AR-siRNA) sequence corresponds to the human AR site 5′-GACUCAGCUGCCCCAUCCA-3′. A NT-siRNA (5′-CCUACGCCACCAAUUUCGU-3′) was used as a control for the siRNA experiments. Following overnight incubation of the suspended cells transfected with varying doses of NT-siRNA or AR-siRNA using Lipofectamine RNAiMAX Reagent (Invitrogen) according to the manufacturer's instructions, media were changed with fresh media and the cells were incubated for the time indicated.

LNCaP, MDA-PCa-2b and LAPC-4 cells were seeded in 6-well plates (1×105/well), grown overnight, and collected after trypsinization. Immediately after 30-min fixation with PBS containing 2% paraformaldehyde, the cells were incubated with murine anti-AR or anti-PSMA mAb in phosphate buffered saline (PBS) containing 1% bovine serum albumin (BSA) and 0.1% saponin (Sigma) for 1 hour, and then the cells were treated with fluorescein isothiocyanate (FITC)-conjugated sheep anti-mouse IgG (H+L, Jackson ImmunoResearch, West Grove, Pa.) antibody for 1 hour. After washing with PBS containing 1% BSA+0.1% saponin, the cells were subjected to fluorescence-activated cell sorting analysis (FACS) (Becton Dickinson, Franklin Lakes, N.J.).

### Immunohistochemical Assessment of PSMA Expression Before and After Castration

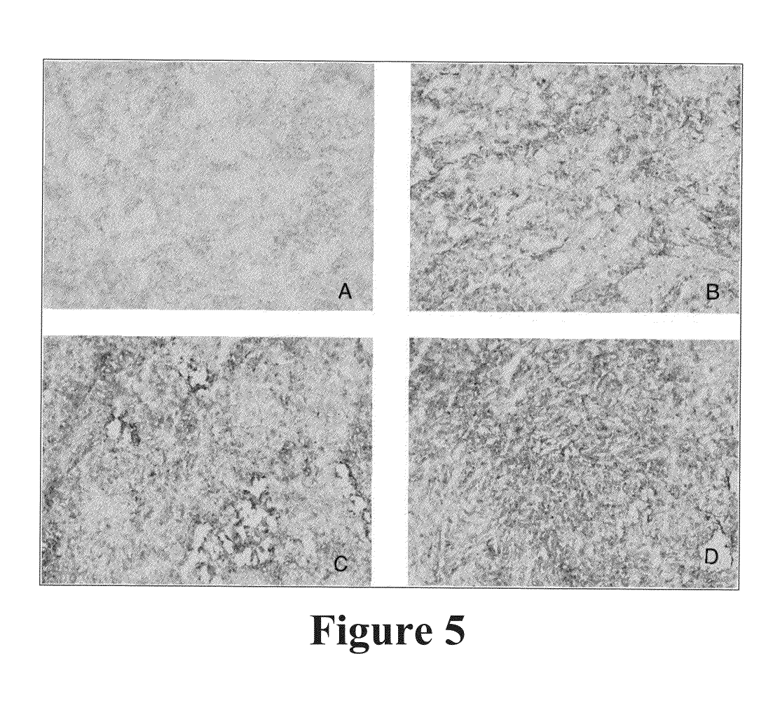

Immunohistochemistry assessment of PSMA expression before and after castration. Baseline PSMA expression of CWR22Rv1 xenograft prior to castration (FIG. 5, panel A). PSMA expression 1 week (FIG. 5 panel B), 2 weeks (FIG. 5 panel C) and 4 weeks (FIG. 5 panel D) post-castration.

CWR22Rv1 xenografts were removed from nude mice. Tumors were pre-cooled in liquid nitrogen, snap-frozen in OCT compound (Sakura Finetek U.S.A., inc., Torrance, Calif.) on dry ice, and stored at −80° C. Cryostat tissue sections were fixed in cold acetone (4° C.) for 10 minutes. The sections were washed in PBS. Peroxidase block (0.03% H₂O₂) was incubated for 5 minutes. After washing in PBS, humanized J591 (10 ug/ml) was incubated on the sections for 1 hour at room temperature. Antibody binding was detected using rabbit anti-human Ig-peroxidase (Dako, Carpinteria, Calif.) secondary antibody and diaminobenzidine (sigma-Aldrich Co., St. Louis, Mo.) as chromogen. The sections were counterstained with 10% Hematoxylin. The diluent (1% bovine serum albumin) was used as negative control.

### DESCRIPTION OF THE PREFERRED EMBODIMENTS

One aspect of the technology is a method of treating prostate cancer in a patient comprising the steps of: (a) assaying a patient's prostate-specific membrane antigen (PSMA) expression; (b) determining, from the assay, if the patients' PSMA expression is indicative of a prostate cancer adenocarcinoma or non-adenocarcinoma; and (c) administering, to the patient, (i) an anti-androgen therapy if the PSMA expression is indicative of an adenocarcinoma or (ii) a chemotherapeutic therapy if the PSMA expression is indicative of a non-adenocarcinoma. In a related aspect, the PSMA expression is assayed by imaging. In another aspect, the imaging is conducted by employing any agent capable of specific binding to PSMA. In a related aspect, the agent is an antibody, antibody derivative, PSMA ligand, small molecule PSMA binder, PSMA enzyme inhibitor, PSMA-binding peptide, or PSMA-binding aptamer. In yet another aspect of the technology, the imaging is done by positron emission tomography (PET), PET/Computed tomography (CT), PET/Magnetic resonance (MR), planar imaging, SPECT imaging, optical imaging, or dye imaging. In one aspect of this technology, the non-adenocarcinoma comprises a prostate small cell, neuroendocrine, or sarcoma.

Another aspect of the technology is a method of treating prostate cancer in a patient comprising the steps of: (a) obtaining a first measurement of a patient's prostate-specific membrane antigen (PSMA) level prior to administering a new prostate cancer therapy with anti-androgen activity; (b) obtaining a second measurement of the patient's PSMA level after administering the new prostate cancer therapy; and (i) continuing the therapy if the second measurement is greater than the first measurement or (ii) discontinuing the therapy if the second measurement is less than or equal to the first measurement. In a related aspect, the time interval between obtaining the first and second measurements is about 2 to 4 weeks. In another aspect, the method further comprises obtaining a third measurement of the patient's PSMA level after administering the new prostate cancer therapy; and (i) continuing the therapy if the third measurement is greater than the first measurement or (ii) discontinuing the therapy if the third measurement is less than or equal to the first measurement. In a related aspect of the technology, the first and/or second measurements of a patient's PSMA level are assayed by imaging. In one aspect, the imaging is conducted by employing any agent capable of specific binding to PSMA. In another aspect, the agent is an antibody, antibody derivative, PSMA ligand, small molecule PSMA binder, PSMA enzyme inhibitor, PSMA-binding peptide, or PSMA-binding aptamer. In one aspect of the technology, the imaging is done by positron emission tomography (PET), PET/Computed tomography (CT), PET/Magnetic resonance (MR), planar imaging, SPECT imaging, optical imaging, or dye imaging.

Yet another aspect of the technology is a method of treating prostate cancer in a patient comprising the steps of: (a) obtaining a measurement of a non-castrated patient's prostate-specific membrane antigen (PSMA) level; and (b) administering, to the patient, a prostate cancer hormonal therapy if the measurement is not elevated and is indicative of normal androgen axis function or seeking an alternative treatment for the patient if the measurement is elevated and is indicative of abnormal androgen axis function. In one aspect, the measurement of a non-castrated patient's PSMA level is assayed by imaging. In another aspect, the imaging is conducted by employing any agent capable of specific binding to PSMA. In yet another aspect, the agent is an antibody, antibody derivative, PSMA ligand, small molecule PSMA binder, PSMA enzyme inhibitor, PSMA-binding peptide, or PSMA-binding aptamer. In a one aspect, the imaging is done by positron emission tomography (PET), PET/Computed tomography (CT), PET/Magnetic resonance (MR), planar imaging, SPECT imaging, optical imaging, or dye imaging.

One other aspect of the technology is a method of treating prostate cancer in a patient comprising the steps of: (a) administering, to a patient, an anti-androgen prostate cancer therapy; and (b) administering, to the patient, an anti-PSMA prostate cancer therapy subsequent to the anti-androgen prostate cancer therapy, thereby producing a synergistic benefit as a result of increasing PSMA density and therefore effect of PSMA-targeted agent. In a related aspect, the time interval between administering the anti-androgen prostate cancer therapy and administering the anti-PSMA prostate cancer therapy is about 2 to 4 weeks.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of treating prostate cancer in a patient comprising the steps of:
(a) assaying a patient's prostate-specific membrane antigen (PSMA) expression;
(b) determining, from the assay, if the patients' PSMA expression is indicative of a prostate cancer adenocarcinoma or non-adenocarcinoma; and
(c) administering, to the patient,
(i) an anti-androgen therapy if the PSMA expression is indicative of an adenocarcinoma or
(ii) a chemotherapeutic therapy if the PSMA expression is indicative of a non-adenocarcinoma.

2. The method of claim 1, wherein the PSMA expression is assayed by imaging.

3. The method of claim 2, wherein the imaging is conducted by employing any agent capable of specific binding to PSMA.

4. The method of claim 3, wherein the agent is an antibody, antibody derivative, PSMA ligand, small molecule PSMA binder, PSMA enzyme inhibitor, PSMA-binding peptide, or PSMA-binding aptamer.

5. The method of claim 2, wherein the imaging is done by positron emission tomography (PET), PET/Computed tomography (CT), PET/Magnetic resonance (MR), planar imaging, SPECT imaging, optical imaging, or dye imaging.

6. The method of any one of claims 1-5, wherein the non-adenocarcinoma comprises a prostate small cell, neuroendocrine, or sarcoma.

7. A method of treating prostate cancer in a patient comprising the steps of:
(a) obtaining a first measurement of a patient's prostate-specific membrane antigen (PSMA) level prior to administering a new prostate cancer therapy with anti-androgen activity;
(b) obtaining a second measurement of the patient's PSMA level after administering the new prostate cancer therapy; and
(i) continuing the therapy if the second measurement is greater than the first measurement or
(ii) discontinuing the therapy if the second measurement is less than or equal to the first measurement.

8. The method of claim 7, wherein the time interval between obtaining the first and second measurements is about 2 to 4 weeks.

9. The method of claim 7, further comprising obtaining a third measurement of the patient's PSMA level after administering the new prostate cancer therapy; and
(i) continuing the therapy if the third measurement is greater than the first measurement or
(ii) discontinuing the therapy if the third measurement is less than or equal to the first measurement.

10. The method of any one of claims 7-9, wherein the first and/or second measurements of a patient's PSMA level are assayed by imaging.

11. The method of claim 10, wherein the imaging is conducted by employing any agent capable of specific binding to PSMA.

12. The method of claim 11, wherein the agent is an antibody, antibody derivative, PSMA ligand, small molecule PSMA binder, PSMA enzyme inhibitor, PSMA-binding peptide, or PSMA-binding aptamer.

13. The method of claim 10, wherein the imaging is done by positron emission tomography (PET), PET/Computed tomography (CT), PET/Magnetic resonance (MR), planar imaging, SPECT imaging, optical imaging, or dye imaging.

14. A method of treating prostate cancer in a patient comprising the steps of:
(a) obtaining a measurement of a non-castrated patient's prostate-specific membrane antigen (PSMA) level; and
(b) administering, to the patient, a prostate cancer hormonal therapy if the measurement is not elevated and is indicative of normal androgen axis function or seeking an alternative treatment for the patient if the measurement is elevated and is indicative of abnormal androgen axis function.

15. The method of claim 14, wherein the measurement of a non-castrated patient's PSMA level is assayed by imaging.

16. The method of claim 15, wherein the imaging is conducted by employing any agent capable of specific binding to PSMA.

17. The method of claim 16, wherein the agent is an antibody, antibody derivative, PSMA ligand, small molecule PSMA binder, PSMA enzyme inhibitor, PSMA-binding peptide, or PSMA-binding aptamer.

18. The method of claim 15, wherein the imaging is done by positron emission tomography (PET), PET/Computed tomography (CT), PET/Magnetic resonance (MR), planar imaging, SPECT imaging, optical imaging, or dye imaging.

19. A method of treating prostate cancer in a patient comprising the steps of:
(a) administering, to a patient, an anti-androgen prostate cancer therapy; and
(b) administering, to the patient, an anti-PSMA prostate cancer therapy subsequent to the anti-androgen prostate cancer therapy, thereby producing a synergistic benefit as a result of increasing PSMA density and therefore effect of PSMA-targeted agent.

20. The method of claim 19, wherein the time interval between administering the anti-androgen prostate cancer therapy and administering the anti-PSMA prostate cancer therapy is about 2 to 4 weeks.
